(19) United States
(12) Patent Application Publication
(10) Pub. No.: US 2013/0289136 A1
(43) Pub. Date: Oct. 31, 2013

(54) SACCHARIDE POLYMER OBTAINED FROM MANIHOT ESCULENTA, PRODUCTION PROCESS, AND USE AS COSMETIC ACTIVE INGREDIENT FOR TIGHTENING THE SKIN

(71) Applicant: Jean Paufique, Objat (FR)

(72) Inventor: Jean Paufique, Objat (FR)

(73) Assignee: SOCIETE INDUSTRIELLE LIMOUSINE D'APPLICATION BIOLOGIQUE, Objat (FR)

(21) Appl. No.: 13/997,590

(22) PCT Filed: Dec. 23, 2011
(86) PCT No.: PCT/FR2011/053182

§ 371 Date: Jun. 24, 2013

(30) Foreign Application Priority Data
Dec. 24, 2010 (FR) 1061257

(51) Int. Cl. A61K 8/84; A61Q 19/08

(52) U.S. Cl. CPC A61K 8/84; A61Q 19/08
USPC 514/772.3; 527/314; 435/95; 435/99

(57) ABSTRACT

A glucan biopolymer obtained from Manihot esculenta as a cosmetic active principle for instant skin tightening, and a cosmetic skincare method using the same. Also, a specific active principle provided in the form of a glucan polymer obtained from Manihot esculenta, the method for obtaining the active principle and the cosmetic compositions including same.

### FIELD OF INVENTION

This invention relates to a cosmetic active ingredient that comes as an articulated glucan polymer that is obtained from Manihot esculenta and to its use on the skin for a tightening effect.

The invention also relates to the process for producing this active ingredient, the cosmetic compositions that include it, and a cosmetic treatment process for a skin-lifting and skin-smoothing effect.

### BACKGROUND OF THE INVENTION

To combat ugly manifestations of cutaneous aging, today there is a broad range of cosmetic products that are to meet requirements for both long-term and immediate results.

This is why, in addition to being endowed with anti-ageing active ingredients that reinforce the cutaneous tissues for permanently reducing the marks of time, these cosmetic care products generally also integrate tightening ingredients, which form a three-dimensional mesh on the skin surface and provide an instant feeling of the effects as well as instantaneous smoothing and lifting rejuvenating surface effects. The skin is thus toned, stretched, and smoothed instantly, and it appears visibly sublimated and younger.

There are several categories of skin-tightening cosmetic agents.

First of all, tightening agents of synthetic polymer type are known, but the latter have numerous disadvantages. They are in particular adhesive and often difficult to formulate because they are only soluble in alcohol.

It is also possible to use certain proteins that are obtained from plant raw materials. These proteins have the advantage of being obtained from natural raw materials and are water-soluble. However, certain cosmetic companies may desire not to use proteins.

More recently, natural carbohydrate-based tightening agents have been developed. The patent FR-2882366 describes in particular cross-linked carbohydrate polymers, obtained by cross-linking reaction on the primary alcohol group of the carbohydrates. However, the active ingredients that are described are not very stable in aqueous medium and therefore cannot be formulated in all of the types of cosmetic compositions.

Furthermore, the patent FR-2908135 also describes a process for the production, starting from natural materials that are rich in carbohydrates, of saccharide polymers that have good tightening effectiveness. However, with certain plant raw materials, the saccharide polymers that are obtained are not physically stable in the presence of conventional cosmetic ingredients, such as certain preservatives (for example, phenoxyethanol) or stabilizers of cosmetic formulas (such as ethylhexylglycerin or pentylene glycol).

There is therefore still a need for a new cosmetic active ingredient that has an instant and instantaneous cutaneous tightening effect, remedying the drawbacks of formulability of the tightening agents of the prior art.

### SUMMARY OF THE INVENTION

To respond to this, this invention proposes using a particular saccharide biopolymer obtained from Manihot esculenta.

In particular, the purpose of the invention is the use of at least one articulated glucan copolymer and copolymerization agents obtained from Manihot esculenta as cosmetic active ingredients having a cutaneous tightening effect.

Advantageously, this saccharide biopolymer has an instant and instantaneous tightening effect. Its structure in the form of an articulated and pre-organized three-dimensional network of high molecular weight is anchored to the skin surface and quickly forms a viscoelastic and film-forming film that is capable of spreading out and adapting perfectly to the microrelief for an instant lifting and smoothing effect.

In addition to its effectiveness, the active ingredient according to the invention can be integrated in a broad range of formulations.

It is recalled that the polymers are substances whose structures result primarily in the repetition of units of low molar mass, called monomers, connected to one another by covalent bonds.

A polymer of natural origin or that is obtained from monomers of natural origin is called “biopolymer” within the scope of this invention.

“Glucan polymer” or “glucan biopolymer” is defined as meaning a glucan copolymer and copolymerization agents (at least one copolymerization agent, preferably two).

“Articulated polymers,” in contrast to linear polymers, are branched polymers. Certain monomers have branched structures or certain monomers have the property of being able to be bonded to at least three other monomers during the polymerization stage. An articulated polymer is therefore a polymer that is formed by elements that can move owing to their bonds. The articulations of the glucan polymer promote the anchoring to the skin surface and the formation of a film-forming viscoelastic film.

The object of the invention is also a specific cosmetic active ingredient that comes in the form of an articulated glucan biopolymer and copolymerization agents obtained from Manihot esculenta as well as its production process.

According to another aspect, the objects of the invention are also cosmetic compositions including between 0.01 and 20% of an articulated glucan biopolymer obtained from Manihot esculenta. These compositions can come in all forms allowing application by topical means.

Finally, the invention also has as its object a cosmetic skincare process, designed to stretch and to smooth the skin instantaneously, comprising the topical application of a composition that contains at least one articulated glucan copolymer and copolymerization agents obtained from Manihot esculenta. This invention is now described in detail.

### DETAILED DESCRIPTION OF THE INVENTION

### Use

According to a first aspect, the purpose of the invention is the cosmetic use of an articulated glucan biopolymer and copolymerization agents obtained from Manihot esculenta as a skin-tightening cosmetic active ingredient.

In particular, the purpose of the invention is the use of an articulated glucan polymer that is obtained from Manihot esculenta as an active ingredient for the production of a cosmetic composition designed to produce an instant tightening effect of the skin.

The particular saccharide polymer according to the invention is capable of being strongly anchored to the skin surface and being spread out there so as to adjust perfectly to the cutaneous microrelief so as to form a continuous and lifting film.

Its adhesion capacities result in particular from the presence of numerous hydroxyl groups on the sugar chains that establish multiple hydrogen bonds with the intercellular lipids of the Stratum corneum, and very particularly like the ceramides, owing to their amide groups and their hydroxyl groups.

The use of an articulated glucan biopolymer that is obtained from Manihot esculenta as a cosmetic active ingredient on the skin therefore makes it possible to quickly form a highly elastic film on the cutaneous surface and thus to provide instantaneous tightening, smoothing and anti-wrinkle effects, both on the face and on the body, on young or mature skin.

Also, in addition to their characteristics of effectiveness, the specific saccharide biopolymers of the invention have the advantage of being able to be incorporated in different types of cosmetic formulas. Actually, they are soluble in water and in ethanol at 20%, they are stable physically in the presence of preservative-type or stabilizer-type cosmetic ingredients, not sensitive to variations of pH and temperature, and they do not have the drawbacks of synthetic polymers, which drawbacks are adhesive and shining effects on the skin.

Also, the purpose of the invention is specifically the use in a cosmetic composition of a cosmetic active ingredient that comes in the form of an articulated glucan polymer obtained from Manihot esculenta, with said active ingredient and/or said composition being designed to smooth the microrelief, fill in the wrinkles, tone and/or stretch the skin.

According to a particularly suitable embodiment, the purpose of the invention is the use of an active ingredient as described below.

### Active Ingredient

The invention also relates to a particular cosmetic active ingredient that comes in the form of an articulated glucan copolymer and copolymerization agents obtained from Manihot esculenta.

The alliance of the inherent properties of Manihot esculenta sugars, in particular glucans, and different specific characteristics of the copolymerization agents makes it possible to obtain an articulated three-dimensional glucan network that is capable of forming on the skin surface a film that is resistant and cohesive but nevertheless endowed with a high flexibility, characterized by significant film-forming and viscoelastic properties.

Actually, the copolymerization agent makes it possible to obtain flexible articulations of the three-dimensional network that impart the tightening, film-forming and spreading-out properties of the polymer. Finally, the degree of copolymerization plays an important role in the resistance and the cohesion of the film.

Preferably, the ratio of the content of molecules obtained from Manihot esculenta relative to the content of copolymerizing agents is greater than 55%.

The active ingredient according to the invention has a mean molecular weight of between values of 7,000 and 300,000 Da, preferably a mean molecular weight of 74,000 Da.

The active ingredient according to the invention can come in the form of clear liquid that is yellow in color. It can be defined by at least one, preferably all, of the characteristics disclosed below.

### Dry Materials

The level of dry materials of an active ingredient according to the invention (measured by running a sample with a given initial weight through the oven at 105° C. in the presence of sand until a constant weight is obtained) can be between 50 and 180 g/l, preferably between 80 and 120 g/l.

### Measurement of pH

The pH (measured by the potentiometric method at ambient temperature) can be between 3.0 and 5.0, preferably between 3.0 and 4.0.

### Carbohydrates

Determination of the Total Sugar Content

The metering of the total sugar content can be implemented by the DUBOIS method (DUBOIS, M. et al. (1956), Analytical Chemistry, 28, No. 3, pp. 350-356). In the presence of concentrated sulfuric acid and phenol, the reducing sugars provide a yellow-orangey compound. Starting from a standard range, it is possible to determine the total sugar level of a sample.

The total sugar level of an active ingredient according to the invention is preferably between 32 and 117 g/l, and even more preferably between 52 and 78 g/l.

The total sugar content can also be expressed in terms of percentage relative to the dry material. It is preferably between 43% and 95%.

### Characterization of Carbohydrates

The characterization of carbohydrates is determined by means of several analyses:
Analysis of the molar mass by HPLC,
Determination of the nature of simple sugars, implemented by ionic liquid chromatography,
Studies of the structure of oligosaccharides by specific enzymatic hydrolysis steps.

The HPLC analysis of molar masses provides the following results:

| | | | Carbo- |
|---|---|---|---|
| | | Degree of | hydrate |
| | Molar Mass (Da) | Polymerization | Level |
| Oligo- | 180 < MM < 7,000 | 1 < DP < 40 | 15% |
| saccharides | | | |
| Poly- | 7,000 < MM < 300,000 | 40 < DP < 1,670 | 85% |
| saccharides | | | |

The composition of simple sugars of the active ingredient determined by ionic liquid chromatography is 100% glucose.

The action of a-amylase releases approximately 40% glucose that is present in the active ingredient according to the invention.

This enzymes cuts the α-1,4 bonds between two glucose molecules. Thus, 40% of the glucose molecules are bonded at α-1,4 only; they do not have branches.

The HPLC analysis shows that after hydrolysis by α-amylase, there are only monosaccharides and disaccharides, corresponding respectively to free glucose and to two glucose molecules bonded at α-1,6, i.e., at the beginning of a branch. The hydrolysis of the α-1,4 bonds by amylase was complete.

The active ingredient according to the invention has:
40% glucose molecules bonded at α-1,4, without a branch,
30% glucose molecules bonded at α-1,4, and α-1,6, i.e., a beginning of a branch,
30% glucose molecules bonded at α-1,6 and corresponding to the first unit of the branch.

The active ingredient according to the invention consists of glucans with a mean molar mass of 74,000 Da. These polysaccharides contain glucose chains bonded at α-1,4 and having branches at α-1,6, approximately 30%.

### Production Process

The active ingredient according to the invention as described above can be obtained by a process comprising at least the series of the following stages:
Solubilization in aqueous phase of Manihot esculenta powder, at a rate of at least 50 g/l,
Optionally, addition of at least one solubilization adjuvant, preferably a salt, a polyphosphate, and/or an oxidizing agent, for facilitating the solubilization of polysaccharides,
Two enzymatic hydrolysis steps, with two different enzymes,
Deactivation of enzymes by heat treatment,
Separation of soluble and insoluble phases, preferably by filtration, decanting, and selective precipitation, for recovering the soluble phase,
Addition of two different copolymerization agents, each having at least one unsaturated ethylene group at a rate of 1 to 200 g/l, preferably 5 to 50 g/l,
Optionally, addition of at least one polymerization initiator for triggering the radical-type copolymerization reaction,
Separation of soluble and insoluble phases, preferably by filtration, by decanting, and by selective precipitation, for recovering the soluble phase,
Purification and concentration of glucan polymers by ultrafiltration, by filtration, and/or reverse osmosis.

The two enzymes that are used for the two successive enzymatic hydrolysis steps are carbohydrases. Preferably, they include α-amylase, β-amylase, glucoamylase, cellulase, amyloglucosidase, β-galactosidase, β-glucanase, inulinase, pectinase, xylanase, arabanase, hemicellulase, rhamnogalacturonase, polygalacturonase, pectinesterase, hemicellulase, and galacturonase.

The hydrolysis stage makes it possible to recover Manihot esculenta glucans.

The two copolymerization agents that are used and that have at least one unsaturated ethylene group are selected from among polyacrylates, polymethacrylates, polyvinyl compounds, and polyallyl compounds.

Preferably, the ratio of the content of molecules obtained from Manihot esculenta relative to the content of copolymerizing agents is greater than 55%.

The polymerization initiator can be selected from among photoinitiators, redox agents and thermal agents.

This process makes it possible to obtain an articulated glucan biopolymer that comes in the form of a three-dimensional network that has an instant skin-tightening effect and that can be easily formulated because of its compatibility with the other components of the cosmetic compositions.

It should be noted that the use of two enzymes and carrying out two enzymatic hydrolysis steps is necessary for the implementation of the process. Actually, tests carried out by the implementation of the process of Example 1 (by changing the hydrolysis stage for each test) starting from two carbohydrases A and B show the importance of carrying out two hydrolysis steps for obtaining a tightening and industrially producible product, i.e., which meets the requirements of formulability imposed for integrating a cosmetic composition (color, liquid, resistance, etc.). The results that are obtained are presented in the table below:

| | Enzymatic | Industrial | Tightening |
|---|---|---|---|
| | Hydrolysis | Feasibility | Effectiveness |
| Product 1 | 1 Hydrolysis Step | Impossible | / |
| | with Carbohydrase A | | |
| Product 2 | 1 Hydrolysis Step | Impossible | / |
| | with Carbohydrase B | | |
| Product 3 | 2 Hydrolysis Steps: | Yes | Yes |
| | 1 with | | |
| | Carbohydrase A and | | |
| | 1 with | | |
| | Carbohydrase B, | | |
| | A/B Ratio = 40/60 | | |

### Cosmetic Compositions and Cosmetic Skincare Process

This invention also covers the cosmetic compositions including at least one articulated glucan biopolymer obtained from Manihot esculenta, in particular an active ingredient as described above, in different galenical forms, adapted to administration by a cutaneous topical method.

These compositions can come in particular in a form that is selected from among the group that consists of an aqueous or oily solution, a cream or an aqueous gel or an oily gel, in particular in a pot or in a tube, in particular a shower gel, a milk, an emulsion, a microemulsion or a nano-emulsion, in particular oil-in-water or water-in-oil either in multiple form or containing silicone, a lotion, in particular in a glass or plastic bottle, or in a metering bottle or aerosol, an ampoule, an ointment, a foam, or an anhydrous product, preferably liquid, pasty or solid.

These compositions are preferably compositions containing between 0.01 and 20% by weight of an active ingredient that comes in the form of an articulated glucan biopolymer obtained from Manihot esculenta, preferably between 1% and 7%.

These compositions comprise, in addition to the active ingredient, a physiologically acceptable and preferably cosmetically acceptable medium, i.e., which does not cause unacceptable sensations of discomfort for the user, such as redness, tingling, or prickling.

The compositions according to the invention can contain as adjuvant at least one compound that is selected from among preservatives, emollients, emulsifying agents, surfactants, moisturizers, thickeners, conditioners, matting agents, stabilizers, antioxidants, texture agents, luster agents, solubilizers, pigments, dyes, perfumes and solar filters. These vehicles are preferably selected from among the group comprising the amino acids and their derivatives, the polyglycerols, the esters, the polymers and derivatives of cellulose, the lanolin derivatives, the phospholipids, the lactoferrins, the lactoperoxidases, the stabilizers that are based on sucrose, vitamin E and its derivatives, natural and synthetic waxes, vegetable oils, triglycerides, unsaponifiables, phytosterols, plant esters, silicones and its derivatives, protein hydrolyzates, liposoluble/water-soluble esters, betaines, aminoxides, plant extracts, saccharose esters, titanium dioxides, glycines, and parabens, and even preferably from among the group that consists of butylene glycol, steareth-2, steareth-21, glycol-15 stearyl ether, cetearyl alcohol, phenoxyethanol, methylparaben, ethylparaben, natural tocopherols, glycerin, sodium dihydroxycetyl, isopropyl hydroxycetyl ether, glycol stearate, triisononanoin, octyl cocoate, polyacrylamide, isoparaffin, laureth-7, a carbomer, propylene glycol, glycerol, bisabolol, dimethicone, sodium hydroxide, PEG 30-dipolyhydroxystearate, capric/caprylic triglycerides, cetearyl octanoate, dibutyl adipate, grape seed oil, magnesium sulfate, EDTA, a cyclomethicone, xanthan gum, citric acid, sodium lauryl sulfate, waxes and mineral oils, isostearyl isostearate, propylene glycol dipelargonate, propylene glycol isostearate, PEG 8 beeswax, lanolin oil, sesame oil, cetyl lactate, lanolin alcohol, ricin oil, titanium dioxide, lactose, saccharose, low-density polyethylene, a salty isotonic solution.

Examples of such adjuvants are cited in particular in the Dictionnaire CTFA (International Cosmetic Ingredient Dictionary and Handbook published by the Personal Care Product Council).

Of course, one skilled in the art will ensure that possible active or non-active complementary compounds and/or their quantities are selected in such a way that the advantageous mixing properties are not, or essentially are not, altered by the addition considered.

Advantageously, the articulated glucan biopolymer that is obtained from the Manihot esculenta according to the invention is stable in aqueous medium and in ethanol at 20%, and it has a very good compatibility and resistance with the above-mentioned adjuvants of the preservative or stabilizer types.

The compositions according to the invention, when they are applied on the skin, make it possible to produce an instant tightening effect. They can be used to smooth the microrelief, fill in wrinkles, tone and/or stretch skin visibly and quickly for an instantaneous impact.

For this purpose, the target of the invention is a cosmetic process for human skincare, designed to smooth the microrelief, fill in the wrinkles, tone and/or stretch the skin, comprising the topical application of a composition that comprises an articulated glucan copolymer and copolymerization agents obtained from Manihot esculenta, in particular with a composition that contains between 0.01 and 20%, and even between 1 and 7% by weight of an active ingredient that comes in the form of an articulated glucan biopolymer that is obtained from Manihot esculenta according to this invention.

### EXAMPLES

### Example 1

A nonlimiting example of the process for producing the active ingredient that comes in the form of an articulated glucan biopolymer obtained from Manihot esculenta is presented below, as well as examples of compositions including such an active ingredient.

An example of a process for producing an active ingredient according to the invention comprises the implementation of the following stages:
Solubilization in aqueous phase of Manihot esculenta powders at a rate of 60 g/l,
Successive enzymatic hydrolysis steps using 2 cellulases at a rate of 0.8% for the first enzyme and 1.2% for the second or vice versa under optimal temperature and pH conditions for the effectiveness of the enzymes,
Deactivation at 70° C. of the enzymatic activities,
Separation of the soluble and insoluble phases by decanting,
Polymerization by adding two copolymerization agents (polyacrylate and polmethacrylate) at a rate of 10 g/l of each agent and a radical-type copolymerization initiator (redox agent),
Separation of the soluble and insoluble phases by decanting,
Purification by filtration and concentration of glucan polymer by successive filtrations.

The active ingredient that is obtained has the following characteristics:
Appearance: clear liquid of a light yellow color
Dry materials: 95.6 g/l
Total sugar content: 67.2 g/l, or 70% relative to the dry materials
Mean molecular weight of the articulated glucan polymer: 74,000 Da.

The mean molecular weight of the copolymer is determined by the HPLC analysis on the following columns: Polymer Laboratories (Varian) PL aquagel-OH 60, aquagel-OH 40 and aquagel-OH 30, and an RI detector. The use of reference molecules makes it possible to calibrate and to evaluate the mean molecular weight of the glucan polymer.

### Example 2

### Use of an Active Ingredient According to the Invention in a Gentle Fluid

| | | |
|---|---|---|
| Phase A. | Water | Enough to |
| | | make 100% |
| Phase B. | Lanol 1688 (Seppic Cetearyl Ethyl- | 10% |
| | hexanoate) | |
| | Montanov 202 (Seppic - Arachidyl | 3% |
| | alcohol/behenyl alcohol/arachidyl | |
| | glucoside/cetyl alcohol) | |
| | Lanol 99 (Seppic-isonyl isonoate) | 2% |
| Phase C. | Preservative | 0.7% |
| | Active ingredient according to this | 4% |
| | invention | |
| Phase D. | Sepigel 305 (Seppic-polyacrylamide, | 2% |
| | C12-14 isoparaffin, laureth-7) | |

The quantities indicated are provided in percentage by weight.

This creamy, white emulsified gel has a pH of 6.7.

It can be obtained by the implementation of the following stages:
Mixing B
Heating A and B separately at 80° C., while being stirred magnetically, for 30 minutes
Emulsifying B in A with an emulsifying rotor-stator at 1,400 rpm for 5 minutes, and then at 1,500 rpm for 30 minutes
Adding C, while being mechanically stirred at 2,000 rpm, continuing to stir for 30 minutes
At 36° C., adding D, while being stirred at 4,000 rpm for 5 minutes, and then at 6,000 rpm for 15 minutes
Removing the emulsified gel, final temperature 39° C.

### Example 3

### Use of an Active Ingredient According to the Invention in a Light Cream

| Phase A. | Water | | Enough to |
|---|---|---|---|
| | | | make 100% |
| | Propylene glycol | | 3% |
| | Glycerol | | 2% |
| Phase B. | Lamol CTO wax | (Seppic) | 2.2% |
| | Cetyl alcohol | (Stéarinerie | 1.6% |
| | | DUBOIS) | |
| | DC 3101 | (Dow Corning) | 2.3% |
| | DUB ININ | (Stéarinerie | 5% |
| | | Dubois) | |
| Phase C. | Micropearl M100 | (Seppic) | 0.6% |
| | MGESVPH | (Faci) | 0.2% |
| | Active ingredient according to the | | 4% |
| | invention | | |
| | Preservative | | 0.7% |
| Phase D. | Citric acid | | Enough to |
| | | | produce pH 6 |

The quantities that are indicated are provided in percentage by weight.

This fluid white emulsion has a pH of 6.

This emulsion can be obtained by the implementation of the following stages:
Heating A and B separately at 80° C. while being mechanically stirred at a low speed,
Emulsifying B in A with an emulsifying rotor-stator at 1,500 rpm,
At 40° C., adding C in the order indicated, while being stirred with a rotor-stator,
At 30° C., adjusting the pH with D slowly while ensuring good dispersion of the powder,
Leaving it under an emulsifying agent until cooling is complete.

### Example 4

### Use of an Active Ingredient According to the Invention in a Light Cream

| Phase A. | Water | | Enough to |
|---|---|---|---|
| | | | make 100% |
| | Glycerol | | 1% |
| Phase B. | DC 345 | (Dow Corning) | 2% |
| | Cetearyl alcohol | (Stéarinerie | 1% |
| | | DUBOIS) | |
| | Montanov 68 | (Seppic) | 5% |
| | DUB BA | (Stéarinerie | 5% |
| | | Dubois) | |
| Phase C. | 2% Satiaxane CX930 | (Degussa) | 10% |
| | Preservative | | 0.7% |

| | |
|---|---|
| Active ingredient according to the | 4% |
| invention | |

The quantities that are indicated are provided in percentage by weight.

This creamy, white creamy gel has a pH of 6.5.

This gel can be obtained by the implementation of the following stages:
Preparing the 2% xanthan gel in water while being mechanically stirred,
Mixing A,
Mixing B,
Heating A and B separately at 80° C. while being stirred,
Emulsifying B in A with an emulsifying rotor-stator at 2,500 rpm,
At 40° C., adding C in order,
Allowing stirring to continue until homogenization is complete.

### Example 5

### Use of an Active Ingredient According to the Invention in a Thick Cream

| Phase A. | Water | | Enough to |
|---|---|---|---|
| | | | make 100% |
| | Glycerol | | 3% |
| | Sataxiane CX 930 | (Degussa) | 0.1% |
| Phase B. | Brij 72 | (Uniquema) | 3.5% |
| | Brij 721 | (Uniquema) | 1.5% |
| | Arlamol E | (Novéon) | 3% |
| | Phytowax Olive 10L40 | (Sophim) | 0.4% |
| | DUB BA | (Stéarinerie | 2% |
| | | Dubois) | |
| | Lanol CTO wax | (Seppic) | 1.5% |
| | Ritaphyl ICS | (RITA) | 7% |
| | DUB PTL | (Stéarinerie | 8% |
| | | Dubois) | |
| Phase C. | Preservative | | 0.7% |

| | |
|---|---|
| Active ingredient according to the | 4% |
| invention | |

The quantities that are indicated are provided in percentage by weight.

This thick white emulsion has a pH of 5.4.

It can be obtained by implementing the following stages:
Mixing A, dispersing the gel thoroughly,
Mixing B,
Heating A and B separately at 80° C. in a water bath, while being mechanically stirred,
Emulsifying B in A in an emulsifying rotor-stator at 1,800 rpm,
Adding C at 40° C.,
Continuing homogenization under an emulsifying agent until cooling is complete.

### Example 6

### Use of an Active Ingredient According to the Invention in a Foundation

| Phase A. | Water | | Enough to |
|---|---|---|---|
| | | | make 100% |
| | Satiaxane CX930 | (Degussa) | 0.2% |
| | Montanox 60 | (Seppic) | 0.4% |
| | Glycerol | | 4% |
| | DUB Diol | (Stéarinerie | 6% |
| | | Dubois) | |
| Phase B. | Simulsol 165 | (Seppic) | 3% |
| | RitaStearic | (RITA) | 1% |
| | Cetyl alcohol | (Stéarinerie | 1% |
| | | Dubois) | |
| | DC345 | (Dow Corning) | 3% |
| | DUB vinyl | (Stéarinerie | 6% |
| | | Dubois) | |
| | Finester EH 25 | (Finetex) | 7% |
| Phase C. | White Covasop | (Sensient LCW) | 6% |
| | W9775 | | |
| | Yellow Covasop | (Sensient LCW) | 1.9% |
| | W1771 | | |
| | Brown Covasop | (Sensient LCW) | 0.4% |
| | W8770 | | |
| | Black Covasop | (Sensient LCW) | 0.3% |
| | W9774 | | |
| Phase D. | Preservative | | 0.7% |

| | |
|---|---|
| Active ingredient according to the | 4% |
| invention | |

The quantities that are indicated are provided in percentage by weight.

This colored, flexible and creamy emulsion has a pH of 5.6.

It can be obtained by implementing the following stages:
Mixing A,
Mixing B,
Heating A and B separately at 80° C. in a water bath, while being mechanically stirred,
Emulsifying B in A with an emulsifying rotor-stator at around 2,000 rpm,
Mixing C until a uniform mixture is obtained,
Adding C at 50° C. into the B/A emulsion, under an emulsifying agent at 2,200 rpm,
Adding D at 40° C.,
Continuing the homogenization under an emulsifying agent until cooling is complete.

### Example 7

### Use of an Active Ingredient According to the Invention in a Sprayable

Lotion

| Phase A. | Water | | Enough to |
|---|---|---|---|
| | | | make 100% |
| | Butylene glycol | | 3% |
| | Propylene glycol | | 4% |
| Phase B. | Sophiderm | (Sophim) | 5% |
| | DUB GMS AE | (Stéarinerie | 1% |
| | | Dubois) | |
| | DC 7 3101 | (Dow Corning) | 13.5% |
| | A72 | (Aiglon) | 2% |
| | DUB PIS | (Stéarinerie | 2% |
| | | Dubois) | |
| Phase C. | Carbopol ETD 2050 | (Novéon) | 0.08% |
| Phase D. | Preservative | | 0.7% |
| | Active ingredient according to the | | 4% |
| | invention | | |
| Phase E. | NaOH | | Enough to |
| | | | produce pH 6.2 |

The quantities that are indicated are provided in percentage by weight.

This white liquid emulsion has a pH of 6.2.

It can be obtained by implementing the following stages:
Mixing A,
Mixing B,
Heating A and B separately at 80° C. in a water bath, while being mechanically stirred,
Emulsifying B in A with an emulsifying rotor-stator at around 2,100 rpm
Adding C and maintaining stirring at around 2,500 rpm for 30 minutes,
Adding D, still under an emulsifying agent and at 2,000 rpm,
Adjusting the pH with E and continuing the homogenization under an emulsifying agent until cooling is complete.

### Example 8

### Use of an Active Ingredient According to the Invention in a Foam Cream

| | | | |
|---|---|---|---|
| Phase A. | Carbopol ETD 2050 | (Novéon) | 0.4% |
| | Water | | 30% |
| Phase B. | Water | | Enough to |
| | | | make 100% |
| | Propylene glycol | | 2% |
| Phase C. | Emulgade 1000 NI | (Henkel) | 1% |
| | Lanette N | (Sidobre) | 1% |
| | Lanol 37 T | (Seppic) | 2% |
| | Arlamol E | (Novéon) | 2% |
| | DUB Aprilose | (Stéarinerie | 2% |
| | | Dubois) | |
| | Sucrose ester SP01C | (Sisterna) | 0.4% |
| Phase D. | Preservative | — | 0.7% |

| | |
|---|---|
| Active ingredient according to the | 4% |
| invention | |

The quantities that are indicated are provided in percentage by weight.

This creamy, foam emulsion has a pH of 6.

It can be obtained by implementing the following stages:
Preparing the gel A, while being mechanically stirred, without adjusting the pH,
Mixing B,
Mixing C,
Heating B and C separately at 80° C. in a water bath, while being mechanically stirred,
Emulsifying C in B with an emulsifying rotor-stator at around 2,000 rpm,
Adding A at 50° C. under an emulsifying agent at 2,500 rpm,
Adding D at 40° C.,
Continuing the homogenization under an emulsifying agent by slowing the stirring little by little until cooling is complete.

### Evaluation of the Effectiveness and of the Formulability of the Active Ingredient According to the Invention

### A. Evaluation of the Effectiveness and of the Formulability of Different Glucan Polymers Obtained from Manihot esculenta and of 2 copolymerization agents

The object is to compare the cosmetic formulability and the tightening effectiveness of copolymers of Manihot esculenta carbohydrates obtained from two copolymerization agents.

The cosmetic formulability is evaluated by observation of the compatibility of the product that is obtained with a conventional cosmetic preservative (phenoxyethanol type).

The cosmetic effectiveness is determined by analysis of the tightening effect.

| | | | Compat- | Tightening |
|---|---|---|---|---|
| | Agent A | Agent B | ibility | Effec- |
| | Level in % | Level in % | Yes/No | tiveness |
| Polymer of | 0 | 100 | No | |
| Manihot esculenta 1 | | | | |
| Carbohydrates | | | | |
| Polymer of | 35 | 65 | No | |
| Manihot esculenta 2 | | | | |
| Carbohydrates | | | | |
| Polymer of | 50 | 50 | Yes | Tightening |
| Manihot esculenta 3 | | | | |
| Carbohydrates | | | | |
| Polymer of | 60 | 40 | Yes | Tightening |
| Manihot esculenta 4 | | | | |
| Carbohydrates | | | | |
| Polymer of | 100 | 0 | Yes | Non- |
| Manihot esculenta 5 | | | | tightening |
| Carbohydrates | | | | |

These results show that the Manihot esculenta carbohydrate biopolymer produced with two copolymerizing agents has a good compatibility with the phenoxyethanol preservative and the desired tightening effectiveness.

### B. Evaluation of the Immediate Effectiveness of a Glucan Biopolymer Obtained from Manihot Esculenta on the Skin

These in-vivo tests have as their object to illustrate the invention by showing the instant effectiveness of an articulated glucan biopolymer that is obtained from Manihot esculenta on the skin.

The active ingredient that is used for these studies is the one of Example 1.

The cosmetic composition that is used in vivo is the one of Example 2.

### I. Evaluation of the Tightening Effect

This study has as its objective to evaluate in vivo vs. a placebo the tightening effect of an active ingredient according to the invention that is formulated at 4% in an emulsified gel for 30 minutes and 1 hour after a single application.

The study was carried out on two groups of healthy female volunteers:
One group for an evaluation at t30 minutes, composed of 18 volunteers with a mean age of 33±6 years,
One group for an evaluation at t1 hour, composed of 18 volunteers with a mean age of 32±5 years.

To evaluate the biomechanical properties of the skin, the method by cutometry was used. Measurements were taken at the level of the forearm using a Cutometer®. Starting from the curves obtained, various characteristic parameters of the mechanical properties of the skin can be calculated. Among these parameters, the following were adopted for quantifying the instant tightening effect:
The parameter Uf (R0), which represents the total elongation: if −Uf increases, the skin is less elastic and therefore more stretched;
The parameter Ue (R7×R0)/R5), which represents instant elasticity: if −Ue increases, the skin is less flexible and therefore more stretched.

The operating procedure of the study is described below.

At t0, before the products are applied, two symmetrical zones are determined at the level of the forearms (one zone treated by placebo and one zone treated by the active ingredient according to the invention).

Measurements with the Cutometer® are taken at these two zones, and the products are applied.

Between t0 and t30 minutes or t1 hour, the volunteers wait in a temperature-controlled and hygrometry-controlled room.

At t30 minutes or t1 hour, after application of the products, measurements are taken again on the corresponding treated zones.

The results that are obtained for the zone that is treated with the active ingredient in percentage of variation relative to the results obtained with the placebo are presented in the table below:

Variation/Placebo (%)

| | −Uf Parameter | −Ue Parameter |
|---|---|---|
| T 30 Minutes | +6.8% | +7.2% |
| T 1 Hour | +5.3% | +7.7% |

It is noted that an active ingredient according to the invention, in comparison to the placebo, 30 minutes and 1 hour after a single application, increases the parameters of the tension of the skin: total elongation and at the same time instant elasticity.

This first study therefore shows well the instant tightening effectiveness of an active ingredient according to the invention that is applied on the skin.

### II. Evaluation of the Smoothing Effect

This study has as its objective to evaluate in vivo vs. a placebo the smoothing effect of an active ingredient according to the invention that is formulated at 4% in an emulsified gel for 30 minutes and 1 hour after a single application.

The study was carried out on two groups of healthy female volunteers:
One group for an evaluation at t30 minutes, composed of 19 volunteers with a mean age of 62±2 years,
One group for an evaluation at t1 hour, composed of 20 volunteers with a mean age of 62±2 years.

The evaluation consists in producing silicone-containing polymer impressions at the level of the forearms before and after treatment, and then in making an analysis by fringe projection (acquisition by volume of the impressions using a fringe projection device dedicated to the 3D measurement of the relief of the impressions). The parameters that are adopted for this study are 3D roughness parameters:
Sq: the root mean square of surface roughness,
Sa: the arithmetic mean of surface roughness.

A reduction of these parameters is characteristic of a smoothing of the surface being studied.

The operating procedure of the study is described below.

At t0, before the products are applied, two symmetrical zones at the level of the forearms are determined (one zone treated by the placebo and one zone treated by the active ingredient according to the invention).

Impressions are made on each of the zones, and the products are applied. Between t0 and t30 minutes or t1 hour, the volunteers wait in a temperature-controlled and hygrometry-controlled room.

At t30 minutes or t1 hour, after the products are applied, impressions on the corresponding treated zones are again produced.

The results that are obtained for the zone that is treated with the active ingredient in a variation percentage relative to the results that are obtained with the placebo are presented in the table below:

Variation/Placebo (%)

| | Sq Parameter | Sa Parameter |
|---|---|---|
| T 30 Minutes | −7.5% | −8.1% |
| T 1 Hour | −5.4% | −5.5% |

It is noted that an active ingredient according to the invention, in comparison to the placebo, 30 minutes and 1 hour after a single application, reduces the 3D roughness parameters that are characteristic of the microrelief of the skin, Sa and Sq. This reduction signifies a smoothing of the microrelief of the skin.

This study therefore shows well the instant smoothing effect of an active ingredient according to the invention that is applied on the skin.

### III. Evaluation of the Anti-Wrinkle Effect

This study has as its objective to evaluate in vivo vs. a placebo the instant anti-wrinkle effect of an active ingredient according to the invention that is formulated at 4% in an emulsified gel for 30 minutes and 1 hour after a single application.

The study was carried out on 17 healthy female volunteers with a mean age of 62±2 years.

The evaluation consists in making 3D acquisitions by fringe projection onto crow's-feet before and after treatment. The parameters that are adopted for this study are:
A 3D roughness parameter: Sa, which corresponds to the arithmetic mean of surface roughness,
A volume parameter: the negative volume that corresponds to the lower volume at the surface of the skin.

A reduction of these two parameters is characteristic of a smoothing of the relief of the surface that is being studied and a reduction of wrinkles.

The operating procedure of the study is described as follows.

At t0, before the products are applied, two symmetrical zones are determined at the level of the crow's-feet (one zone that is treated by the placebo and one zone that is treated by the active ingredient according to the invention).

Acquisitions are made of each crow's-foot by fringe projection, and the products are applied.

Between t0 and t30 minutes or t1 hour, the volunteers wait in a temperature-controlled and hygrometry-controlled room.

At t30 minutes or t1 hour, after the products are applied, acquisitions of each crow's-foot are again made by fringe projection.

The results that are obtained for the zone that is treated with the active ingredient in percentage of variation relative to the results that are obtained with the placebo are presented in the table below:

Variation/Placebo (%)

| | Sa Parameter | Negative Volume |
|---|---|---|
| T 30 Minutes | −6.2% | −11.0% |
| T 1 Hour | −10.2% | −16.9% |

It is noted that an active ingredient according to the invention, in comparison to the placebo, 30 minutes and 1 hour after a single application, smoothes and minimizes the wrinkles by reducing both the surface roughness (Sa) and the negative volume.

This study therefore shows well the instant anti-wrinkle effect of an active ingredient according to the invention that is applied on the skin.

### C. Influence of Different Parameters on the Effectiveness of a Glucan

Biopolymer Obtained from Manihot Esculenta on the Skin

Tests have also been carried out for evaluating the possible influence of different parameters on the effectiveness of the active ingredient according to the invention: the time, the metering, the treated zone, the formulation, and the age.

The active ingredient that is used for these studies is that of Example 1.

The cosmetic composition that is used in vivo is that of Example 2.

### I. Influence of Time

### I.1. Study of the Instant Tightening Properties by Cutometry

The objective of this study is to evaluate in vivo vs. a placebo the influence of time on the tightening effect of an active ingredient according to the invention that is formulated at 4% in emulsified gel.

The measurements were taken at the level of the forearms using a Cutometer® 30 minutes, 1 hour, 2 hours, and 4 hours after a single application.

The study was carried out on four groups of healthy female volunteers:
One group for an evaluation at 30 minutes, composed of 18 volunteers with a mean age of 33±6 years,
One group for an evaluation at 1 hour, composed of 18 volunteers with a mean age of 32±5 years,
One group for an evaluation at 2 hours, composed of 18 volunteers with a mean age of 34±5 years,
One group for an evaluation at 4 hours, composed of 17 volunteers with a mean age of 35±5 years.

The operating procedure is the one described in Item B.I/.

The results that are obtained for the −Uf and −Ue parameters are summarized in the table below:

Variation/Placebo (%)

| | −Uf Parameter | −Ue Parameter |
|---|---|---|
| T 30 Minutes | +6.8% | +7.2% |
| T 1 Hour | +5.3% | +7.7% |
| T 2 Hours | +7.1% | +10.9% |
| T 4 Hours | +5.6% | +9.2% |

It is noted that the active ingredient according to the invention has a significant tightening effect as soon as 30 minutes after a single application. This effect reaches its maximum 2 hours after the application and is again significant after 4 hours.

### I.2. Study of the Instant Anti-Wrinkle Properties by Fringe Projection

The objective of this study is to evaluate in vivo vs. a placebo the influence of time on the anti-wrinkle effect of an active ingredient according to the invention that is formulated at 4% in an emulsified gel for 5 minutes, 30 minutes, 1 hour and 2 hours after a single application.

3D acquisitions by fringe projection onto crow's-feet have been carried out before and after treatment.

The study was carried out on 17 healthy female volunteers with a mean age of 62±2 years.

The operating procedure is the one that is described in Item B.III/.

The results that are obtained for the parameters Sa and negative volume are summarized in the table below:

Variation/Placebo (%)

| | Sa Parameter | Negative Volume |
|---|---|---|
| T 5 Minutes | −5.2% | −11.0% |
| T 30 Minutes | −6.2% | −11.0% |
| T 1 Hour | −10.2% | −16.9% |
| T 2 Hours | −10.4% | −15.6% |

It is noted that the active ingredient according to the invention tends to reduce the characteristic parameters of the cutaneous relief as soon as 5 minutes after a single application and becomes significant after 30 minutes. This effect reaches its maximum 1 hour after application and extends up to 2 hours in a significant way.

### II. Influence of the Metering

### Study of the Instant Tightening Effect by Cutometry

The objective of this study is to evaluate in vivo vs. a placebo the influence of the active ingredient dose according to the invention that is formulated with emulsified gel on the tightening effect.

The measurements were taken at the level of the forearms using a Cutometer® 1 hour after a single application.

The study was carried out on three groups of healthy female volunteers:
One group for a 2% dose, composed of 17 volunteers with a mean age of 38±3 years,
One group for a 4% dose, composed of 18 volunteers with a mean age of 32±5 years,
One group for a 7% dose, composed of 17 volunteers with a mean age of 38±3 years.

The operating procedure is the one described in Item B.I/.

The results that are obtained for the −Uf and −Ue parameters are summarized in the table below:

Variation/Placebo (%)

| | −Uf Parameter | −Ue Parameter |
|---|---|---|
| 2% Dose | +3.8% | +6.8% |
| 4% Dose | +5.3% | +7.7% |
| 7% Dose | +6.1% | +11.3% |

It is noted that the active ingredient according to the invention has a significant instant tightening effect upon 2% of use and that this effect increases with the dose. Under the conditions of this study, it reaches its maximum at 7%.

### III. Influence of the Treated Zone

### Study of the Instant Tightening Properties by Cutometry

The objective of this study is to evaluate in vivo vs. a placebo the instant tightening effect of an active ingredient according to the invention that is formulated at 4% in emulsified gel.

The measurements were taken on different cutaneous zones (forearms and face) using a Cutometer^{(R)} 1 hour after a single application.

The study was carried out on two groups of healthy female volunteers:
One group for the forearms, composed of 18 volunteers with a mean age of 32±5 years,
One group for the face, composed of 22 volunteers with a mean age of 32±6 years.

The operating procedure is the one described in Item B.I/. The results that are obtained for the −Uf and −Ue parameters are summarized in the table below:

Variation/Placebo (%)

| | −Uf Parameter | −Ue Parameter |
|---|---|---|
| Forearms | +5.3% | +7.7% |
| Face | +7.2% | +7.7% |

It is noted that the active ingredient according to the invention is capable of increasing the characteristic parameters of the tension of the skin on the face and on the body, 1 hour after a single application.

### IV. Influence of the Formulation

### Study of the Instant Tightening Properties by Cutometry

The objective of this study is to evaluate in vivo vs. a placebo the influence of the formulation on the instant tightening effect of an active ingredient according to the invention.

The measurements were taken at the level of the forearms using a Cutometer® 1 hour after a single application.

The study was carried out on three groups of healthy female volunteers:
One group for a gel formula composed of 17 volunteers with a mean age of 33±5 years,
One group for an emulsified gel formula composed of 18 volunteers with a mean age of 32±5 years,
One group for an emulsion formula composed of 17 volunteers with a mean age of 33±5 years.

The formulas that are used are the following:

### Gel Formula:

| | |
|---|---|
| Active Ingredient According to the Invention | 4.0% |
| Acrylate C10-30 Alkyl Acrylate Crosspolymer | 0.5% |
| (Carbopol ETD2020, Novéon) | |
| Preservatives | 0.7% |
| NaOH | Enough to |
| | produce |
| | pH 6.5 |
| Water | Enough to |
| | make 100% |

Emulsified Gel Formula:

| | |
|---|---|
| Cetearyl Ethylhexanoate (Lanol 1688 Seppic) | 10.0% |
| Active Ingredient According to the Invention | 4.0% |
| Arachidyl Alcohol/Behanyl Alcohol/Arachidyl | 3.0% |
| Glucoside (Montanov 20, Seppic) | |
| Isonyl Isonoate | 2.0% |
| (Lanol 99, Seppic) | |
| Polyacrylamide/C13-14 Isoparaffin/Laureth-7 | 2.0% |
| (Sepigel 305, Seppic) | |
| Preservatives | 0.7% |
| Water | Enough to |
| | make 100% |

Emulsion Formula:

| | |
|---|---|
| Isonyl Isonoate (Lanol 99, Seppic) | 27% |
| Cetearyl Ethylhexanoate (Lanol 1688, Seppic) | 20% |
| Active Ingredient According to the Invention | 4.0% |
| Cetearyl Alcohol/Cetearyl Glucoside (Montanov | 3.0% |
| 68, Seppic) | |
| Preservatives | 0.7% |
| Water | Enough to |
| | make 100% |

The operating procedure is the one described in Item B.I/.

The results that are obtained for the −Uf and −Ue parameters are summarized in the table below:

Variation/Placebo (%)

| | −Uf Parameter | −Ue Parameter |
|---|---|---|
| Gel | +5.5% | +5.9% |
| Emulsified Gel | +5.3% | +7.7% |
| Emulsion | +3.1% | +6.8% |

It is noted that the active ingredient according to the invention is capable of increasing in a significant way the characteristic parameters of the tension of the skin 1 hour after a single application, in different types of cosmetic formulas.

### V. Influence of Age

### Study of Instant Anti-Wrinkle Properties by Fringe Projection

The objective of this study is to evaluate in vivo vs. a placebo the instant anti-wrinkle effect of an active ingredient according to the invention that is formulated at 4% in emulsified gel on different age groups in comparison to the placebo, 1 hour after a single application.

3D acquisitions by fringe projection of crow's-feet were carried out before and after treatment.

The study was carried out on two groups of healthy female volunteers:
One “Young Skin Panel,” composed of
   Active ingredient group composed of 21 volunteers with a mean age of 42±5 years, and
   Placebo group composed of 18 volunteers with a mean age of 41±5 years
A “Mature Skin Panel” group testing the active ingredient and the placebo, composed of 17 volunteers with a mean age of 62 ±2 years.

The operating procedure is the one that is described in Item B.III/.

The results that are obtained for the parameters Sa and Negative Volume are summarized in the table below:

Variation/Placebo (%)

| | Sa Parameter | Negative Volume |
|---|---|---|
| Young Skin Panel | −6.0% | −14.1% |
| Mature Skin Panel | −10.2% | −16.9% |

It is noted that the active ingredient according to the invention significantly reduces the characteristic parameters of the wrinkles of the skin 1 hour after a single application regardless of the age of the volunteers.

The results of these different tests therefore show that the active ingredient according to the invention does indeed have a quick and significant tightening effect at 30 minutes and 1 hour after its application and that is still significant at least 4 hours afterwards. This tightening effect is dose-dependent, and measurable on the face and the body, where it is formulated in a gel, emulsified gel, or in an emulsion.

The active ingredient according to the invention also has instant smoothing and anti-wrinkle properties that make it possible to visibly fill in wrinkles on young and mature skin.

### D. Evaluation of the Formulability of a Glucan Biopolymer Obtained from

### Manihot Esculenta on the Skin

These tests have as their objective to show the resistance and the good compatibility of an active ingredient according to the invention with cosmetic raw materials. The active ingredient that is used is the one of Example 1.

### I. Study of pH Resistance

A study of pH resistance was carried out at 20° C. for a range of pH varying between 2 and 10.

The results that are obtained are presented below:

| Active Ingredient | | | | | | | | | |
|---|---|---|---|---|---|---|---|---|---|
| According to the Invention | pH | | | | | | | | |
| (v/v) | 2 | 3 | 4 | 5 | 6 | 7 | 8 | 9 | 10 |
| 1% | + | + | + | + | + | + | + | + | + |
| 2.5% | + | + | + | + | + | + | + | + | + |
| 5% | + | + | + | + | + | + | + | + | − |

+: stable
−: unstable

It is noted that the active ingredient according to the invention remains stable and that it is not sensitive to pH variations.

### II. Study of Temperature Resistance

A study of temperature resistance was carried out at the pH of the solution for temperatures ranging from 40 to 80° C. for 2 hours.

| Active Ingredient | Time-Temperature Pair | | | | | | | | |
|---|---|---|---|---|---|---|---|---|---|
| According to the | 40° C. | | | 60° C. | | | 80° C. | | |
| Invention (v/v) | 30′ | 60′ | 120′ | 30′ | 60′ | 120′ | 30′ | 60′ | 120′ |
| 1% | + | + | + | + | + | + | + | + | + |
| 2.5% | + | + | + | + | + | + | + | + | + |
| 5% | + | + | + | + | + | + | + | + | + |

+: stable
−: unstable

It is noted that the active ingredient according to the invention remains stable and that it is not sensitive to temperature variations.

### III. Study of Ethanol Resistance

A study of solubility in different water/ethanol mixtures was done at 20° C. and at the pH of the solution.

The table below summarizes the compatibility of the active ingredient according to the invention with ethanol.

| Active Ingredient | | | | | | | |
|---|---|---|---|---|---|---|---|
| According to the | Ethanol/H₂O (v/v) | | | | | | |
| Invention (v/v) | 10/90 | 20/80 | 30/70 | 40/60 | 50/50 | 60/40 | 70/30 |
| 1% | + | + | + | − | − | − | − |
| 2.5% | + | + | + | − | − | − | − |
| 5% | + | + | − | − | − | − | − |

+: stable
−: unstable

It is noted that the active ingredient according to the invention is stable at least up to 20% ethanol.

### IV. Study of Resistance Relative to Cosmetic Raw Materials

A study was carried out to estimate the resistance of 5% of an active ingredient according to the invention relative to the raw materials used in cosmetics.

The results are presented below:

| | |
|---|---|
| Cosmetic Raw Materials | Resistance |

Thickener

| | |
|---|---|
| 0.5% Carbopol TEA enough to | ++ |
| produce pH = 6.5 | |
| (ETD 2020 - Noveon) | |
| 2% Sepigel 501 | ++ |
| (Seppic) | |
| 2% Xanthan | ++ |
| (Satia xane CX 930 - Evonik) | |
| 3% Cellulose Gum | ++ |
| (Blanose 9M31F - Aqualon) | |
| 4% Carraghenane | ++ |
| (Satiagel CT 52 - Evonik) | |
| 3% Quaternized Cellulose (Polyquat | ++ |
| 400 - RITA) | |

Emulsifying agent

| | |
|---|---|
| 5% Nonionic | ++ |
| (Montanov 202 - Seppic) | |
| 5% Anionic | ++ |
| (Stearic Acid/TEA) | |
| 5% Cationic | ++ |
| (Amonyl 380 BA - Seppic) | |

++ Total compatibility of the active ingredient and the raw material (absence of precipitation, fluidification)
+ The active ingredient entrains a slight fluidification of the formula
− Incompatible (liquefaction, precipitation)

It is noted that the active ingredient according to the invention is stable relative to the cosmetic raw materials that are commonly used.

### V. Study of Resistance in Different Formulations

Finally, a study has also been carried out to estimate the resistance of an active ingredient according to the invention in different cosmetic formulas.

The active ingredient according to the invention has been introduced at 5% in the following cosmetic formulas:

| Clear Gel: | | Opaque Gel: | |
|---|---|---|---|
| Carbopol Ultrez 10 | 0.5% | Sepigel 305 | 3% |
| NaOH | Enough to | Lanol 99 | 25% |
| | produce | Preservative | 0.7% |
| | pH = 6.3 | Water | Enough to |
| Glycerol | 10% | | make 100% |
| Propylene glycol | 10% | | |
| Preservative | 0.7% | | |
| Water | Enough to | | |
| | make 100% | | |
| Emulsified Gel: | | Non-Ionic Emulsion: | |
| Montanov 202 | 3% | Montane 60 | 2% |
| Isopropyl Palmitate | 10% | Montanox 60 | 4% |
| Preservative | 0.7% | Isopropyl Palmitate | 20% |
| Sepigel 305 | 2% | Preservative | 0.7% |
| Lanol 1688 | 2% | Water | Enough to |
| Water | Enough to | | make 100% |
| | make 100% | | |
| Anionic Emulsion: | | Cationic Emulsion: | |
| Stearic Acid | 7% | Amonyl DM | 5% |
| Triethanolamine | Enough to | Cetearylic Alcohol | 1% |
| | produce | Gemseal 60 | 8% |
| | pH = 8 | Cetyl Alcohol | 1% |
| Ritaphyl ICS | 20% | PEG 100 Stearate | 1% |
| Preservative | 0.7% | Preservative | 0.7% |
| Water | Enough to | Water | Enough to |
| | make 100% | | make 100% |

The results that express the physical resistance of these different formulas containing 5% of an active ingredient according to the invention are presented in the table below. The resistance is characterized by an absence of precipitation of the active ingredient, an absence of creaming or phase shift of the formula.

| Cosmetic Formulas | Resistance |
|---|---|
| Aqueous Solution | + |
| Clear Gel | + |
| Opaque Gel | + |
| Emulsified Gel | + |
| Non-Ionic Emulsion | + |
| Anionic Emulsion | + |
| Cationic Emulsion | + |

+ stable
− unstable

These results show that the active ingredient is stable in different cosmetic formulations.

These different tests therefore show that the tightening cosmetic active ingredient according to the invention, in addition to its effectiveness, also has performances in terms of formulability: it is stable in water and in ethanol at 20%, it is not sensitive to variations in pH and in temperature, and it is compatible with the materials that are commonly used in cosmetics.

1-18. (canceled)

19. A cosmetic active ingredient comprising an articulated glucan copolymer and copolymerization agents obtained from Manihot esculenta with a mean molecular weight of between 7,000 and 300,000 Da.

20. The cosmetic active ingredient according to claim 19, wherein the mean molecular weight is 74,000 Da.

21. The cosmetic active ingredient according to claim 19, wherein said cosmetic active ingredient has at least one characteristic selected from the group consisting of:
a dry material of between 50 and 180 g/l,
a pH of between 3.0 and 5.0, and
a total sugar level of between 32 and 117 g/l.

22. The cosmetic active ingredient according to claim 19, wherein said cosmetic active ingredient has at least one characteristic selected from the group consisting of:
a dry material of between 80 and 120 g/l,
a pH of between 3.0 and 4.0, and
a total sugar level of between 52 and 78 g/l.

23. The cosmetic active ingredient according to claim 19, wherein the articulated glucan copolymer contains glucose chains bonded at α-1,4 with branches at α-1,6.

24. A process for the production of the cosmetic active ingredient according to claim 19, comprising the steps of:
solubilizing Manihot esculenta powder in an aqueous phase, at a concentration of at least 50 g/l;
performing two enzymatic hydrolysis steps on said solubilized Manihot esculenta,
deactivating enzymes from said enzymatic hydrolysis steps by heat treatment;
separating soluble and insoluble phases after said heat treatment and recovering the soluble phase;
polymerizing by adding two different copolymerization agents to the soluble phase, each having at least one unsaturated ethylene group in order to glucan polymers,
separating soluble and insoluble phases after polymerizing and recovering the soluble phase,
purifying and concentrating glucan polymers formed from the soluble phase recovered after polymerizing.

25. The process for the production of a cosmetic active ingredient according to claim 24, wherein the enzymes are carbohydrases that are selected from the group consisting of: α-amylase, β-amylase, glucoamylase, cellulase, amyloglucosidase, β-galactosidase, β-glucanase, inulinase, pectinase, xylanase, arabanase, hemicellulase, rhamnogalacturonase, polygalacturonase, pectinesterase, hemicellulase, and galacturonase.

26. The process for the production of a cosmetic active ingredient according to claim 24, wherein the two different copolymerization agents that are used, having at least one unsaturated ethylene group, are selected from the group consisting of polyacrylates, polymethacrylates, polyvinyl compounds and polyallyl compounds.

27. The process for the production of a cosmetic active ingredient according to claim 24, wherein the polymerizing step further comprises adding a polymerization initiator selected from the group consisting of photoinitiators, redox agents and thermal agents.

28. The process for the production of a cosmetic active ingredient according to claim 24, wherein the two different copolymerization agents, each having at least one unsaturated ethylene group, are added in a concentration of 1 to 200 g/l.

29. A cosmetic composition comprising a glucan copolymer and copolymerization agents obtained from Manihot esculenta as a skin-tightening cosmetic active ingredient.

30. The cosmetic composition according to claim 29, wherein the glucan copolymer obtained from Manihot esculenta has a mean molecular weight of between 7,000 and 300,000 Da.

31. The cosmetic composition according to claim 29, wherein the active ingredient produces an instant tightening effect of the skin.

32. The cosmetic composition according to claim 29, wherein the active ingredient smooths microrelief, fills in the wrinkles, tones and/or stretches skin.

33. A cosmetic composition for topical application, comprising an active ingredient according to claim 19, present between 0.01 and 20% by total weight of the composition.

34. A cosmetic composition for topical application, comprising an active ingredient according to claim 19, present between 1 and 7% by total weight of the composition.

35. A cosmetic process for smoothing microrelief, filling in wrinkles, toning and/or stretching skin of a subject, comprising topically applying to a subject in need thereof a composition that comprises a glucan copolymer and copolymerization agents obtained from Manihot esculenta.

36. The cosmetic process according to claim 35, where the subject is human and the glucan copolymer obtained from Manihot esculenta has a mean molecular weight of between 7,000 and 300,000 Da.
